(19) United States
(12) Patent Application Publication
(10) Pub. No.: US 2003/0232451 A1
(43) Pub. Date: Dec. 18, 2003

(54) Device for the testing of fluid samples and process for making the device

(76) Inventors: Douglas Casterlin, Kinderhook, NY (US); Stan Cipkowski, Kinderhook, NY (US)

Correspondence Address: Edmund M. Jaskiewicz, Suite 400, 1730 M Street NW, Washington, DC, 20036, US

(21) Appl. No.: 10/602,859

(22) Filed: Jun. 25, 2003

Related U.S. Application Data
(63) Continuation-in-part of application No. 08/981,665, filed on Nov. 5, 1997.
Application No. 08/981,665 is a continuation-in-part of application No. 08/613,487, filed on Mar. 11, 1996, now Pat. No. 5,976,895.

(51) Int. Cl. G01N 33/558

(52) U.S. Cl. 436/514

(57) ABSTRACT

A diagnostic test device has a thin flat core member having at least one groove indented into its face and an immunoassay strip is seated in each of the grooves. Physical projections in the form of nibs are on the opposed longitudinal edges of each groove for retaining a test strip in its groove. In a process for making such an immunoassay diagnostic test device the thin flat core member is molded from a high impact resistant plastic material and grooves are indented into a face of the core member. During molding, projecting points are formed on both edges of the groove but spaced apart such a distance to enable a test strip to be pushed there-between. The drug device is assembled by pushing a test strip past the projecting points onto the bottom of the groove such that the strip is retained in the groove by the projecting points. A test card front label may be then applied to the face of the core to enclose the test strips and appropriate openings are provided in the panel to provide access to the test strips and to provide for viewing of the visual test results.

### RELATED APPLICATIONS

This is a continuation-in-part application of U.S. patent application Ser. No. 08/981,665 filed Nov. 5, 1997 which is a continuation-in-part of application Ser. No. 08/613,487 filed Mar. 11, 1996.

### TECHNICAL FIELD

The present invention relates to the assaying and testing of fluid samples for drugs of abuse, more particularly, to a diagnostic test device for indicating visually the presence of particular drugs of abuse in urine and a process for making the test card.

### BACKGROUND ART

The increased availability and use of drugs of abuse by the general population has caused employers, governmental agencies, sports groups and other organizations to utilize drug screening both as a condition of employment and in order to maintain safety in the workplace. Typical drug screening tests are performed for the purpose of quickly identifying on a qualitative basis the presence of drugs in a body fluid which may be urine. A complete analysis of the sample may then be carried out in a laboratory only if the preliminary screening results are positive. More and more such drug screenings are taking place on site or the workplace and are generally carried out by testing personnel who may not be technically trained, such as laboratory technicians. It is thus important that the drug screening procedure is simple but yet reliable. Further, the test apparatus must be such so as to enable the testing personnel to avoid all contact with the fluid specimen which is being tested.

Various forms of devices which have been proposed for the collection and taking of body fluids, such as urine, have proved to be cumbersome in operation since they involve a number of separate steps. Initially, the sample was collected and several additional steps were then required to transfer the urine sample to an analysis device. This multiple step procedure required the manual handling of the specimen through various devices and the use of such transfer devices inevitably caused spills which may result in contamination to the tester and surroundings. In addition, non technical personnel who perform the screening tests on urine samples objected to coming into any kind of contact with the urine sample and even the handling of the sample itself.

Many of the known testing systems were rather complex in that they included a container for the specimen, and, subsequently it was necessary to transfer the specimen or at least a portion thereof to another compartment of the container in order to perform the test. This transfer of the specimen required vigorious shaking of the container or turning the container upside down in order to cause the flow of the specimen into a test compartment. It was therefore necessary to make the containers leak proof under such conditions and the result was a complicated and expensive container structure.

The testing system also included a screen test card for drugs of abuse which comprised a thin flat member having the size and shape of a business card. A plurality of immunoassay test strips are fastened side by side in parallel on at least one side of the test card within the outline of the card. Each test strip is reactive to provide a visual indication in response to a particular drug of abuse.

The test card comprised a central ply of styrene which had a thickness corresponding to or slightly greater than the thickness of the test strips and slots are provided in the center ply to receive the test strips. The top and bottom faces of the central ply are covered by a bottom ply and a top ply. The top and bottom plys may be of a thin vinyl sheet or cardboard coated with plastic. The top ply is provided with a plurality of test windows through which the test results as indicated by the test strips can be seen. At the lower or or bottom end of the card are provided sample openings through which the liquid test specimen is able to contact the absorbant or sample receiving portion of the test strips.

A test card could be modified to have test strips on both sides thereof to increase the number of different drugs which can be tested while maintaining the same overall size and configuration of the test card.

The test card was insertable into a cup-like container so as to have one end immersed in the urine sample retained in the container to a predetermined depth whereby the visual results of each test strip can be seen through a transparent wall of the container or above the container without removing the test card from the container. Each test strip is reactive to provide a visual indication in response to a particular drug of abuse. The test card thus provides for the simultaneous detection of multiple analytes. If the sample should test “positive” to indicate the presence of a drug in the urine it is then necessary to send the sample to a certified laboratory for confirmatory testing. The rest card had test strips spaced apart in parallel on a test side of the test card and extending longitudinally of the test card from top to the bottom thereof.

### DISCLOSURE OF INVENTION

It is therefore the principal object of the present invention to provide a novel and improved test device or card having a plurality of immuno-assay test strips on one or both sides thereof with each strip being responsive to a particular drug of abuse and having a visual end point to indicate the presence or absence of a particular drug.

It is an additional object of the present invention to provide a simple and effective process for making such a drug test device.

The objects of the present invention are achieved and the disadvantages of the prior art are eliminated by the diagnostic test device according to the present invention which may comprise a thin flat core member having at least one groove indented into a face thereof and an immuno-assay test strip in each of the grooves. Means are provided on the longitudinal edges of said groove for retaining a test strip which has been seated in the groove. The retaining means may comprise a plurality of nibs projecting from opposed edges of a groove and these nibs are so spaced on the groove edges to enable a test strip to be inserted there-between into the groove. The grooves are preferably parallel on one or both faces of the core member. A front panel member may then be positioned over the core to enclose the test strips therein and this panel has openings therein to provide access to the test strip and to view the test results indicated visually thereon.

A process for making the diagnostic test device according to the present invention may comprise the steps of molding a thin flat core member from a high impact resistant plastic material and forming a groove indented into a face of the core member and further forming projecting points on both edges of a groove spaced apart such a distance to enable a test strip to be pushed there-between. A test strip is then pushed past the projecting points onto the bottom of the groove such that the strip is secured seated within the groove by the projecting points.

The grooves and the projecting points on the grooves are preferably formed in a single molding step. A front panel member may then be applied upon the core to enclose the test strips. Openings are formed in the front panel to provide access to the strips and to review the test results indicated visually on the strips.

### BRIEF DESCRIPTION OF THE DRAWINGS

Other objects and advantages of the present invention will be apparent upon reference to the accompanying description when taken in conjunction with the following drawings, which are exemplary, wherein;

FIG. 1 is a perspective view of the drug abuse test kit according to the present invention generally showing the container, the test card partially inserted to the testing position in the container through a slit in the cover;

FIG. 2 is an exploded perspective view of the container according to the present invention for collecting and testing a fluid sample and generally showing the container, a cover having a slit covered with a removable adhesive seal and a second solid closure cap;

FIG. 3 is a plan view of the test side of a test card according to the present invention;

FIG. 4 is a plan view of the reverse side of the test card shown in FIG. 3;

FIG. 5 is an end elevational view of the test card shown in FIG. 3;

FIG. 6 is a sectional view taken along the line VI-VI of FIG. 3;

FIG. 7 is a plan view of another modification of the test card;

FIG. 8 is a sectional view taken along the line VIII-VIII of FIG. 7;

FIG. 9 is a plan view of the test side of a further modification of the test card;

FIG. 10 is a plan view of the center ply of the test card of FIG. 9 and showing a test strip in a slot thereof;

FIG. 11 is a plan view of the reverse side of the test card of FIG. 9;

FIG. 12 is a sectional view taken along the line XII-XII of FIG. 9;

FIG. 13 is a plan view of the test side of a molded test card core according to the present invention;

FIG. 14 is a sectional view taken along the line XIV-XIV of FIG. 13;

FIG. 15 is a plan view in enlarged scale of detail D on FIG. 13;

FIG. 16 is a sectional view similar to that of FIG. 14 but showing grooves on both faces of the test card core;

FIG. 17 is a plan view of a modification of a test card core shown in FIG. 13 in which a single recess is provided to accommodate a plurality of test strips in side by side relationship;

FIG. 18 is a sectional view taken along the line XVIII-XVIII in FIG. 17;

FIG. 19 is a plan view of a plurality of test strips assembled in side by side relationship;

FIG. 20 is a side elevational view of the assembly of strips shown in FIG. 19.

FIG. 21 is a schematic view illustrating the assembly of the drug test device using a jig or template.

### MODES FOR CARRYING OUT THE INVENTION

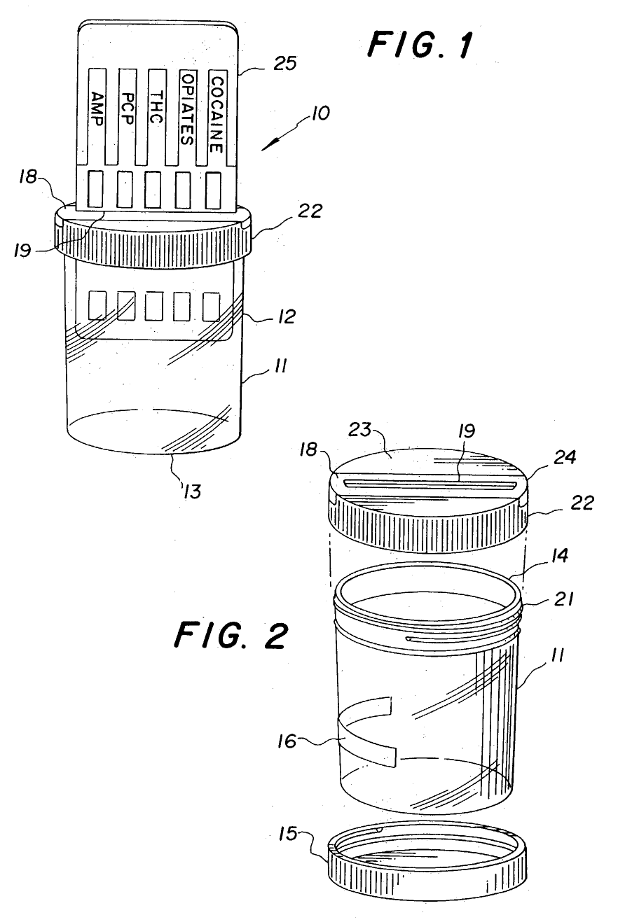

As may be seen in FIGS. 1 and 2, a drug abuse test kit is indicated generally at 10 and comprises a cup-like transparent test container or cup 11 having a cylindrical side wall 12, a closed bottom 13 and an open top 14. The cylinderical wall 12 may have a slight taper or be straight.

The open end 14 of the test cup 11 is provided with external threads 21 upon which is seated an outer closure cover or cap 22 provided with corresponding internal threads which are not shown in the drawing. The cover 22 has a circular top surface 23 from the periphery of which depends a cylindrical wall 24 on the inner surface of which there are provided internal threads. The cover surface 23 has a diametrical slit 19 therein shaped to accommodate a test card as will be presently described. There is also provided a solid cover or cap 15 which is similar in size and shape to the cover 22 but is solid or unslit so that the covers 15 and 22 may be interchangeably mounted on the open end 14 of the test card 11. During shipment, the cover 15 is generally fitted on the bottom of the test cup. A temperature strip 16 is mounted on the bottom side wall of the test cup so as to be responsive to the temperature of the test sample within a test cup.

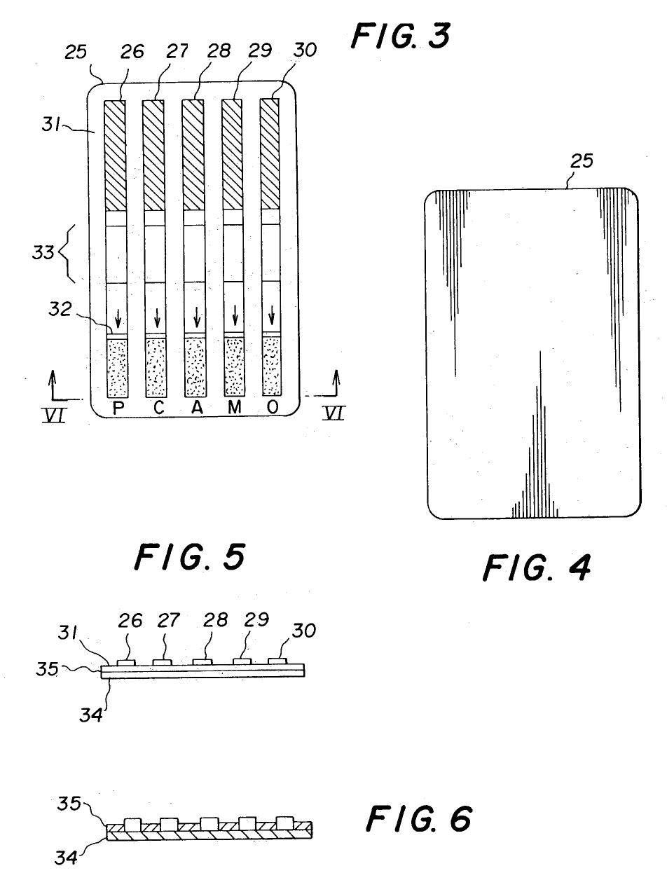

A test card 25 which will indicate the presence or absence of any one of five different drugs of abuse is shown in FIG. 1 inserted within the slit 19 in the closure cap 22 and in further detail in FIGS. 3-6. A test card is of the multiple drug type in that test strips for five different drugs of abuse are mounted on the test card. The test strips 26-30 are spaced apart in parallel on a test side 31 of the test card. These test strips indicate the presence or absence of the following specific drugs of abuse: PCP, cocaine, amphetamines (AMP), marijuana (THC) and opiates. Test strips 26-30 may be of the type as made by Phamatech of San Diego, Calif. and Arista Biological of Bethlehem, Pa. Such test strips are characterized as immunoassay strips and employ colloidal gold chemistry. Each test strip is submerged up to a maximum line indicated at 32 and the results of the test are read in a test area indicated at 33. A blue line in the test area indicates positive or the presence of the particular drug in the test sample.

The test strips are actually recessed in slots in the card so that portions of the test strips project above the test surface 31 of the card as seen in FIG. 5. The test strips are placed into the slots as shown in FIG. 6 and each of the test strips is adhered to the surface of the first portion 34 upon which the second portion 35 has been attached.

It is also within the scope of this invention to make this test card of two separate or individual plys 34 and 35 which are then adhered together and the strips are fixed in the slots as described above.

in order to conduct a drug abuse test utilizing the test card according to the present invention a person being tested must first provide a urine sample into the transparent test cup 11. The quantity of specimen provided must be such as to permit insertion of the test card up to about the maximum line indicated at 32. It is also possible to provide fill lines on the wall surface of the test container.

The test cup with a sufficient quantity of test specimen therein is then closed by threading the cap 22 on the top of the test cup. The cap 22 is provided with a readily removable adhesive sealing strip 18 which is placed over the slit 19. Thus, when the container with the test specimen is brought to the person conducting the test, the protective strip 18 is removed and the multiple drug test card 25 inserted into the slit so that the bottom of the test card rests upon the bottom of the test cup. Thirty to forty-eight (30-48) ml. of specimen will insure that a sample receiving portion at the bottom end of the strip will be in contact with the specimen. The level of this quantity of fluid will be slightly below or above the fill line 32. The test card then remains in place for at least three minutes and the results of the test can be read on each individual test strip through the transparent wall of the container. Thus, if a blue line appears on any one of the test strips, this indicates positive and the presence of that particular drug of abuse in the test specimen. If no such blue line appears then the absence of any of the five drugs of abuse from the specimen is indicated. With such a negative result, the urine sample and the container are discarded.

However, when the results of the test are positive. It is preferable to send the specimen to a certified laboratory for a confirmatory analysis by more specific methods of testing such as gas chromatography or mass spectrometry. In order ship the sample in the container, the closure 22 is removed and the solid cover 15 is threaded down tightly upon the open end of the container.

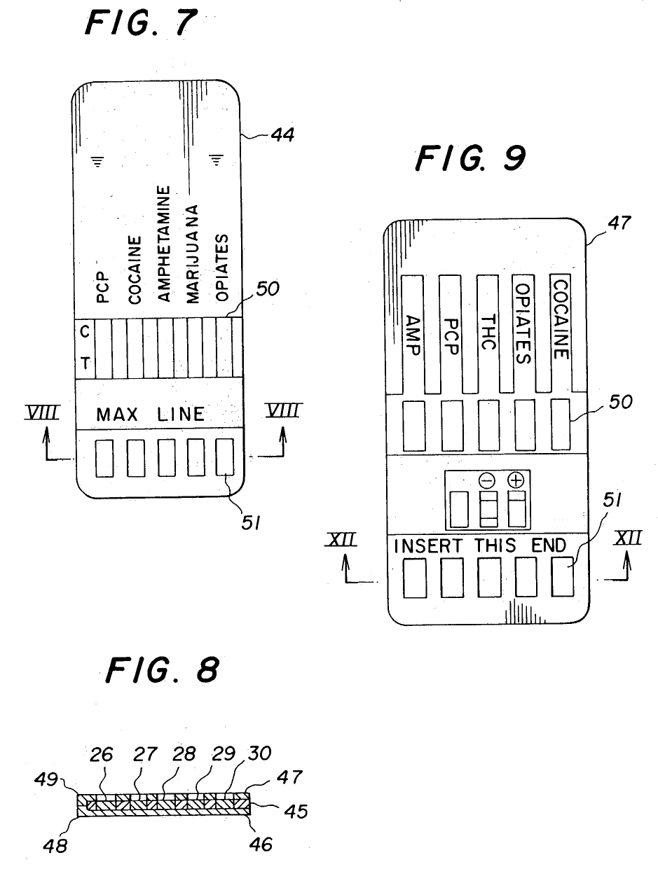

A modification of the test card is shown at 44 in FIG. 7. In this modification, the test strips are covered but the pertinent test and sample portions of the test strips are exposed through openings. The test card 44 comprises a central ply 45 of styrene which has a thickness of 1.25 mm. Corresponding to or slightly greater than the thickness of the test strips and slots are provided in the center ply to receive the test strips. The top and bottom faces of the central ply 45 are covered by a bottom ply 46 and a top ply 47 which may be made from a single piece of material double scored at 48 and 49 so as to wrap around the central ply 45 in the manner as shown in FIG. 8. The top and bottom plys may be of a thin vinyl sheet or cardboard coated with plastic. The top ply 47 is provided with a plurality of test windows 50 through which the test results as indicated by the test strip can be seen. At the lower end of the card are provided sample openings 51 through which the liquid test specimen is able to contact the absorbent or sample portion of the test strips.

In FIGS. 9-12 there is shown a modification of the test card 44 in which the card is made of three separate plys which are then laminated. The bottom and top plys 46 and 47 are made of a thin vinyl sheet having a thickness of 0.33 mm. and the center ply 45 is made of styrene having a thickness of about 1.25 mm. The top ply 47 similarly has the test openings or windows 50 and the sample openings 51 and the bottom ply 46 is solid as shown.

The center ply 45 is provided with a plurality of longitudinal extending slots 52 and a test strip 53 is seated in each of these slots as shown. The test strip generally has a length less than that of the slot 52. In this embodiment, only a single test strip for THC (marijuana) is shown. While this embodiment of the test card has provision for five test strips, it is to be understood that the card can be made in the same manner with less than five strips and even a single strip if so desired. In such a modification, the windows 50 and 51 for the omitted strips are usually solid.

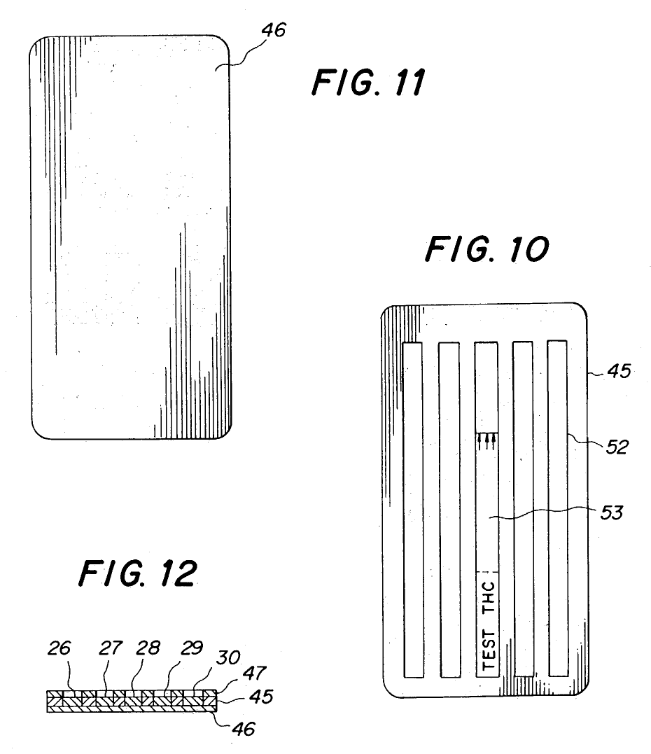

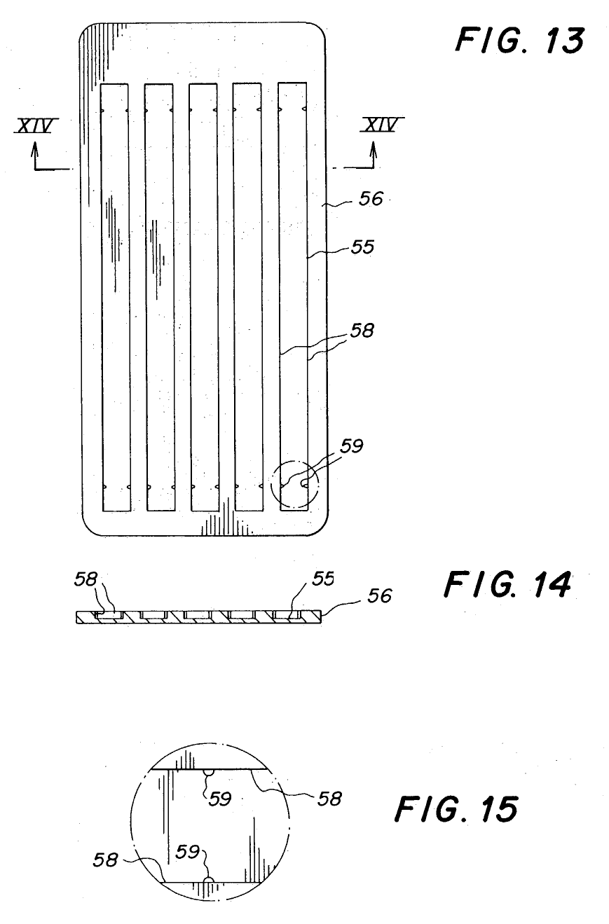

In FIG. 13, there is shown a thin flat test device core 54, rectangular in shape, which is similar to the center ply 45 of the test card shown in FIG. 10. The test device core 54 similarly has a plurality of longitudinally extending grooves 55 indented into a face 56 of the test device core 54. Each groove has a base or bottom 57 and a pair of opposed longtudinal side walls 58. Each groove has a pair of opposed projecting points or nibs 59 projecting into the groove from opposed side walls approximate the ends of the grooves as shown in FIG. 13. The nibs are each preferably arcuate in shape and are spaced below the face 56 of the test device a distance of the order of 0.003 inches. Each nib projects into its respective groove a distance of the order of 0.1 inches. Thus, the distance between opposing nibs is such to enable a test strip 53 to be pushed or snapped into place within a groove but the nibs will then retain the test strips seated into the groove without an adhesive.

Each test device 54 together with one or more indented grooves and nibs at the edges of the grooves is molded from a high impact polystyrene (HIPS), in particular, such a plastic material known as NOVA IMPACT PS 5711 which has favorable density, melt-flow rate, rigidity and shrink characteristics and is used in thin-walled and medical packaging, house-hold goods and toys. The test device 54 is then molded from a mold in a conventional manner as known in the art.

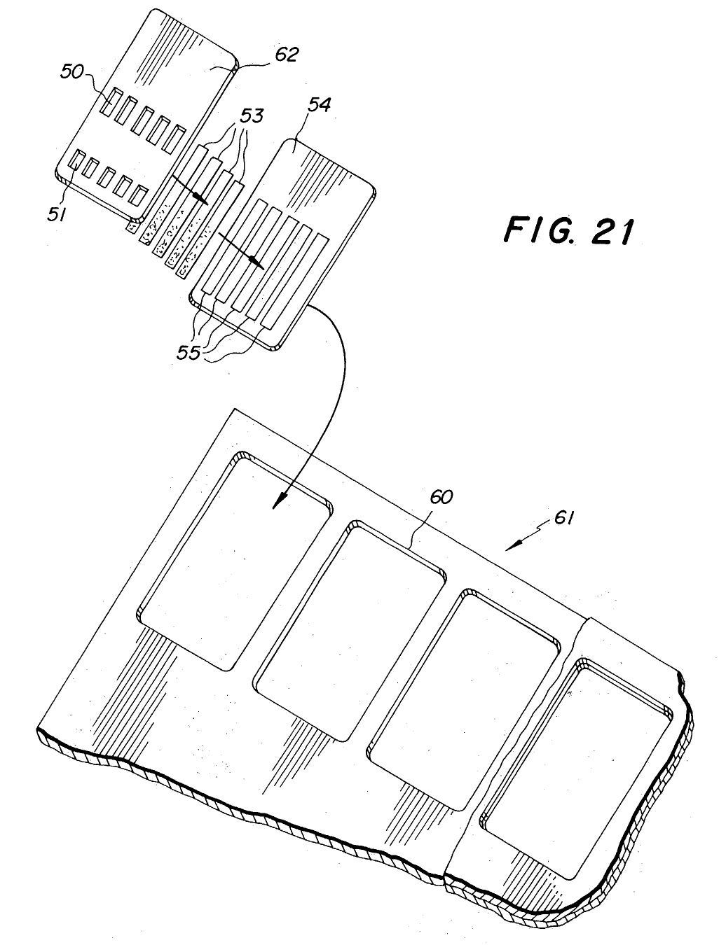

In order to assemble a diagnostic test device using the molded core 54 disclosed above, the core 54 is initially placed in the proper orientation in an appropriately configured recess 60 in an assembly jig indicated generally at 61 in FIG. 21.

One or more appropriate test strips 53 are then inserted into grooves 55 and pushed down firmly on the two areas of each test strip corresponding to the nibs on the groove. This action will “snap” the test strip into place in the core seated on the bottom of its respective groove.

An appropriate test card front label or panel 62 may then be applied to the face of the core. This label may be adhesive backed covered by a release paper which is removed immediately prior to application. The front panel 62 may be formed with the test and sample receiving windows 50 and 51 as shown or the windows 50 and 51 can be extended to interconnect so that a single opening is formed.

It is to be noted that there is no adhesive to secure the test strip in its groove. The “snapping” of the test strip past the pairs of nibs into place in a groove is necessary because of the lack of an adhesive coated surface.

Thus, the molded core eliminates the need for a bottom or back ply such as 46 shown above which covers the bottom face of a slotted central ply as shown at 45. As a result, assembly of a test card using a molded core requires less time since the assembly process is simplified. It is estimated that the resulting increase in efficiency and productivity is about ten percent.

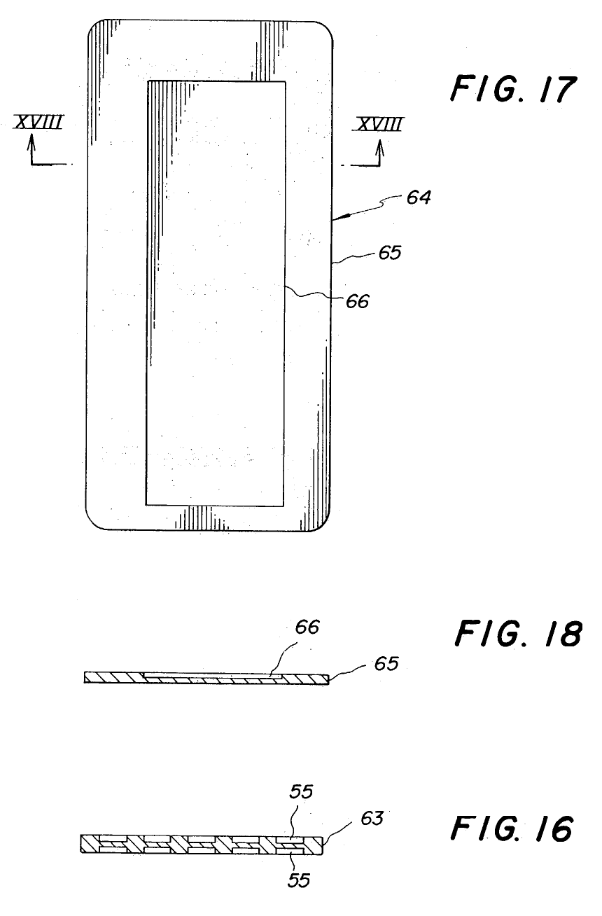

The test card core can also be molded with grooves on both sides of the core as shown at 63 in FIG. 16 in order to carry a greater number of test strips, each of which tests for a different drug of abuse. Each side of such a test core will be the same as shown in FIG. 13 but FIG. 16 shows in section such a double-sided test card core.

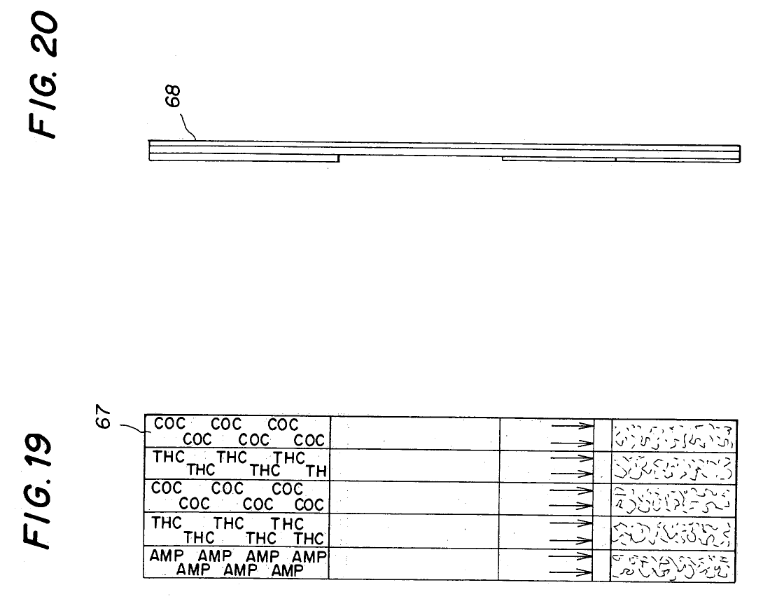

A modification of the diagnostic test device 54 is shown in FIG. 17 at 64 in which the test card core 65 is molded with a single recess 66 indented into one or both faces of the core 65. The recess is shaped to accommodate a plurality of test strips 67 as shown in FIG. 19 which may be mounted upon a thin rigid plastic backing member 68 in contiguous side-by-side relationship without any spaces between individual test strips. A longitudinal side wall of the recess 66 are similarly provided with opposed pairs of nibs 69 which retain the assembly of test strips 67 seated within the recess 66.

Tests conducted to date have indicated that the immunoassay test strips used in the drug test device will function properly and effectively when they are positioned in contacting side-by-side relationship and will provide test results similar in accuracy to test cards having spaced test strips thereon.

The nibs in the side of the grooves thus provide a physical obstruction to the removal, accidental or deliberate, of the test strips from their respective grooves.

Each of the test strips 26-30 is a one-step immunoassay in which a specially treated drug, (drug conjugate) competes with a drug which may be present in the sample specimen for the limited number of binding sites on an antibody. The test strip consists of a membrane strip onto which a drug conjugate has been immobolized. A collodial gold-antibody complex is dried at one end of the membrane. In the absence of any drug in the urine sample, the colldial gold-antibody complex moves with the urine sample by capillary action to contact the immobilized drug conjugate. An antibody-antigen reaction occurs forming a visible line in the test area of the test strip. The formation of a visible line occurs when the test is negative for the drug. When a drug is present in the urine sample, the drug or its metabolite will compete with the immobolized drug congate in the test area for the limited antibody sites on the colldial gold-labeled antibody complex. If a sufficient amount of drug is present, it will fail all of the available binding sites, thus preventing attachment of the label antibody to the drug congate. An absence of a color line or band in the test area is indicative of a positive result. A control band or line comprised of a different antibody/antigen reaction is present on the membrane strip. The control line is not influenced by the presence or absence of drug in the urine and therefore should be present in all reactions.

In summary, if a single band appears in the control zone and no band appears in the test zone, then the results are “positive” which indicates that that particular drug is present above a predetermined level which is usually around 50 ng/ml. If two color bands appear, one in the control region and the other in the test region, then the rest results are “negative” which indicates that the level of that particular drug is below the predetermined detection of sensitivity.

In the event there are no distinct color bands visible in both the test zone and the control zone or if there is a visible band in the test zone but not in the control zone, then the result is invalid and testing of the specimen is recommended with another test card.

The test card can also be used as a carrier or delivery system for a biological detection or monitor device by replacing the drug test strips with strips treated with suitable chemicals so as to be responsive to different and selected biological warfare agents. The strips will then function similarly to drug abuse strips to provide visual indication of the presence in a predetermined quantity of a specific biological warfare agent or the absence of such an agent.

Thus it can be seen that the present invention discloses a novel and improved drug test device which comprises a multiple drug test card which is inserted in the specimen within a container and the visual results of the test may be read on the test card through the transparent wall of the container. The test card comprises a number of individual test strips of the immunoassay type and each strip is responsive or indicative to a particular drug of abuse or to a specific infectious disease. The test card may be made of a core of molded plastic and a thin sheet of plastic or cardboard which may be, laminated upon the core. This drug abuse test kit enables one to obtain rapidly a visual, qualitative result which is very advantageous for forensic purposes but is not limited to such purposes.

It will be understood that this invention is susceptible to modification in order to adapt it to different usages and conditions, and accordingly, it is desired to comprehend such modifications within this invention as may fall within the scope of the appended claims.

What is claimed is:

1. A diagnostic test device comprising a thin flat core member having at least one groove indented into a face thereof, an immunoassay test strip in a said groove, and means on the edges of each of said grooves for retaining said test strip in its respective groove.

2. A diagnostic test device as claimed in claim 1 wherein said means for retaining are on opposed longitudinal edges of a said groove.

3. A diagnostic test device as claimed in claim 1 wherein said means are so spaced apart on said groove edges to enable a test strip to be inserted there-between.

4. A diagnostic test device as claimed in claim 1 wherein said core is molded from a plastic material.

5. A diagnostic test device as claimed in claim 1 wherein said means for retaining comprise a plurality of nibs projecting from opposed longitudinal edges of a said groove.

6. A diagnostic test device as claimed in claim 1 wherein there is a plurality of spaced parallel grooves in a face of said core member.

7. A diagnostic test device as claimed in claim 6 wherein there are grooves in both faces of said core member.

8. A diagnostic test device as claimed in claim 1 wherein said groove has sufficient width to accommodate a plurality of test strips in side-by-side relationship.

9. A diagnostic test device as claimed in claim 8 wherein said plurality of test strips are attached together.

10. A diagnostic test device as claimed in claim 9 wherein said test strips are attached together on a backing sheet.

11. A diagnostic test device as claimed in claim 1 and further comprising a front panel positioned over the core member to enclose the test strips therein and having openings to provide access to the test strip and to review test results.

12. A process for making a diagnostic test device comprising the steps of molding a thin flat core member from a high impact resistant plastic material and indenting a groove into a face of the core member and further forming projecting points on both edges of the groove but spaced apart such a distance to enable a test strip to be pushed there-between, and pushing a test strip between said projecting points onto the bottom of the groove such that the strip is secured in the groove by the projecting points.

13. A process as claimed in claim 12 wherein said grooves and projecting points are formed in a single molding step.

14. A process as claimed in claim 12 and the additional step of applying a front panel member upon the core member to enclose the test strips.

15. A process as claimed in claim 14 and forming at least one opening in the front panel member to provide access to the test strip and review of the test results thereon.
